(19) United States
(12) Patent Application Publication
(10) Pub. No.: US 2019/0029547 A1
(43) Pub. Date: Jan. 31, 2019

(54) VITAL SIGN SIGNAL PROCESSING DEVICE

(71) Applicant: SHARP KABUSHIKI KAISHA, Sakai City, Osaka (JP)

(72) Inventors: YUICHI WATARAI, Sakai City (JP); EIJI SUEMATSU, Sakai City (JP); KEISUKE SATOH, Sakai City (JP)

(21) Appl. No.: 16/073,498

(22) PCT Filed: Jan. 5, 2017
(86) PCT No.: PCT/JP2017/000088

§ 371 Date: Jul. 27, 2018

(30) Foreign Application Priority Data
Jan. 27, 2016 (JP) 2016-013043

(51) Int. Cl. A61B 5/024; A61B 5/08; A61B 5/00

(52) U.S. Cl. CPC A61B 5/02444; A61B 5/0816; A61B 5/7203; A61B 5/05; A61B 5/7278; A61B 5/6892; A61B 5/7221

(57) ABSTRACT

A vital sign signal processing device having an excellent detection accuracy of a vital sign signal is provided. A vital sign signal processing device includes: a plurality of vital sign signal detection units configured to detect vital sign signals of a subject; and a signal processing unit configured to process the vital sign signals detected by the plurality of vital sign signal detection units and to acquire vital sign information denoting a vital phenomenon which is movement of a living body. Each of the plurality of vital sign signal detection units includes a transmission unit configured to transmit a radio wave with which the living body is irradiated and a reception unit configured to receive a signal corresponding to a radio wave reflected off the living body, and each of the plurality of vital sign signal detection units is configured to detect the vital sign signal from the signal received by the reception unit.

### TECHNICAL FIELD

The present invention relates to vital sign signal processing devices, and specifically, to a vital sign signal processing device configured to detect and process a vital sign signal of a subject. This application claims the priority benefit of Japanese Patent Application No. 2016-013043 filed with the Japan Patent Office on Jan. 27, 2016, the content of which is incorporated herein by reference in its entirety.

### BACKGROUND ART

Irradiating a measuring object with an electromagnetic wave to use the Doppler shift of a reflected wave reflected off the measuring object is a widely known method for obtaining a vibrational state and/or displacement of the measuring object. An electromagnetic wave in the microwave to millimeter-wave band also has a property of penetrating through a medium such as a dielectric, and thus, using the electromagnetic wave in an attempt to detect a pulsation of the heart and/or respiration appearing as a vibration in a human body (subject) by irradiating the subject with the microwave is recently proposed. Using the microwave enables measurement without contact with the human body and with the subject wearing clothes, which reduces stress of the subject during sensing. An example of a sensing device by using such a microwave is a vital sign signal sensing device disclosed in PTL 1 (Japanese Unexamined Patent Application Publication No. 2010-120493).

### CITATION LIST

### Patent Literature

PTL 1: Japanese Unexamined Patent Application Publication No. 2010-120493

### SUMMARY OF INVENTION

### Technical Problem

A conventional microwave sensor device has the following problems to be solved.

Firstly, depending on a measurement environment such as a positional relationship between a subject and a measurement device, noise influences vital sign information represented by, for example, a heart rate, a respiratory rate, and the presence or absence of body motion, which leads to degradation in sensing accuracy.

Such degradation in sensing accuracy is usually such that the heart rate has an accuracy of about ±10% of the actual numerical value and the respiratory rate has an accuracy of about ±5% of the actual numerical value. However, depending on the distance and/or positional relationship between the subject and the measurement device, a numerical value largely deviating from the accuracy may be output instantaneously. Moreover, in an attempt to sense body motion, there may be a case where a signal is buried in noise, and thus, the presence or absence of the body motion cannot be detected.

Secondly, the conventional microwave sensor device performs sensing by a single flat-panel antenna and thus has a narrow sensing range, and adjusting the range is difficult.

The sensing range of the heartbeat and/or respiration by the conventional single antenna has an azimuth angle and an elevation angle each having 30 degrees and a distance of about 1 meter (body motion can be sensed in about 10 meters). Thus, an installation location of the antenna and a sensing range required for adjustment of the angle have to be secured. The small sensing range is also a factor that causes the degradation in sensing accuracy.

It is an object of the present invention to provide a vital sign signal processing device having an excellent detection accuracy of a vital sign signal.

### Solution to Problem

A vital sign signal processing device according to an aspect of the present disclosure includes: a plurality of vital sign signal detection units configured to detect vital sign signals of a subject; and a signal processing unit configured to process the vital sign signals detected by the plurality of vital sign signal detection units and to acquire vital sign information denoting a vital phenomenon which is movement of a living body. Each of the plurality of vital sign signal detection units includes a transmission unit configured to transmit a radio wave with which the living body is irradiated and a reception unit configured to receive a signal corresponding to a radio wave reflected off the living body, and each of the plurality of vital sign signal detection units is configured to detect the vital sign signal from the signal received by the reception unit.

Preferably, each vital sign signal includes a heartbeat signal denoting a heartbeat or a respiratory signal denoting respiration, the vital sign information includes heart rates or respiratory rates, and the signal processing unit is configured to discard a heart rate or a respiratory rate of the heart rates or the respiratory rates acquired from the vital sign signals which exceeds a threshold value.

The signal processing unit is preferably configured to calculate a central value of the heart rates or the respiratory rates acquired from the vital sign signals.

Preferably, the signal processing unit is configured to perform comparison of pattern information denoting a pattern of a predetermined change of vital sign information of the subject with the vital sign information acquired from the vital sign signals which are detected so as to determine body motion which is body movement of the subject in accordance with the comparison.

Preferably, the vital sign signal detection unit is configured to detect the vital sign signals in time sequence, and the signal processing unit is configured to determine a time when the body motion occurs in time sequence in accordance with the comparison.

The plurality of vital sign signal detection units preferably perform wireless or wired communication with the signal processing unit.

### Advantageous Effects of Invention

According to the present disclosure, vital sign signals detected by a plurality of vital sign signal detection units are used to improve the detection accuracy of vital sign signals.

### BRIEF DESCRIPTION OF DRAWINGS

FIG. 1 is a view schematically illustrating an arrangement of a vital sign signal processing device 100 according to a first embodiment.

FIG. 2 is a view schematically illustrating an arrangement of the vital sign signal processing device 100 according to the first embodiment.

FIG. 3 is a view schematically illustrating a configuration in a housing 101 of the vital sign signal processing device 100 of FIG. 1.

FIG. 4 is a view schematically illustrating attachment of a vital sign signal processing device including a plurality of vital sign signal detection units according to a second embodiment.

FIG. 5 is a view schematically illustrating irradiation ranges (see shaded areas in the figure) of radio waves of vital sign signal detection units 30A to 30E of FIG. 4.

FIG. 6 is a view schematically illustrating connection of the vital sign signal processing device 100 and a display unit 90.

FIG. 7 is a view illustrating an internal configuration of vital sign signal detection units 30 according to a third embodiment in connection with peripheral elements.

FIG. 8 is a view illustrating a configuration of a signal processing unit 40 of FIG. 7.

FIG. 9 is a graph illustrating an example of a body motion waveform signal 71b according to a fourth embodiment.

FIG. 10 is a block diagram illustrating a signal processing unit 40 according to a fifth embodiment in connection with peripheral elements.

FIG. 11 is a flow chart illustrating processes performed by the signal processing unit 40 according to the fifth embodiment.

### DESCRIPTION OF EMBODIMENTS

Vital sign signal processing devices of embodiments of the present invention will be described below with reference to the drawings. Note that in the drawings referred hereinafter, portions denoted by the same reference signs have the same functions, and thus, the description thereof is not repeated unless needed.

[Outline]

In each embodiment, a vital sign signal processing device 100 includes: a plurality of vital sign signal detection units; and a signal processing unit 40 configured to process the vital sign signals detected by the plurality of vital sign signal detection units and to acquire vital sign information denoting a vital phenomenon which is movement of a living body. Each of the plurality of vital sign signal detection units is configured to irradiate a living body with a radio wave, receive a reflected wave corresponding to the radio wave reflected off the laying body, and detect the vital sign signal from a reception signal corresponding to the reflected wave.

Thus, the vital sign signals are detected in accordance with the reception signals received by the respective vital sign signal detection units. Therefore, it is possible to avoid a situation that the vital sign signal cannot be detected due to noise. Moreover, irradiation with a plurality of radio waves enables a larger detection range to be secured.

In each embodiment, vital sign signal detection units 30A to 30E have identical configurations. Thus, when being mentioned collectively, the vital sign signal detection units 30A to 30E are referred to as vital sign signal detection units 30. Moreover, the vital phenomenon denotes movement, such as a pulsation (heartbeat) of the heart or respiration, appearing in a living body. The vital sign signal is a signal obtained by measuring the vital phenomenon by, for example, a sensor and includes a signal such as a waveform convertible into a numerical value. Moreover, the body motion denotes body movement of a subject and includes, for example, movement of the chest and rolling over of the body.

### First Embodiment

A vital sign signal processing device 100 according to a first embodiment includes a housing and a plurality of vital sign signal detection units accommodated in the housing.

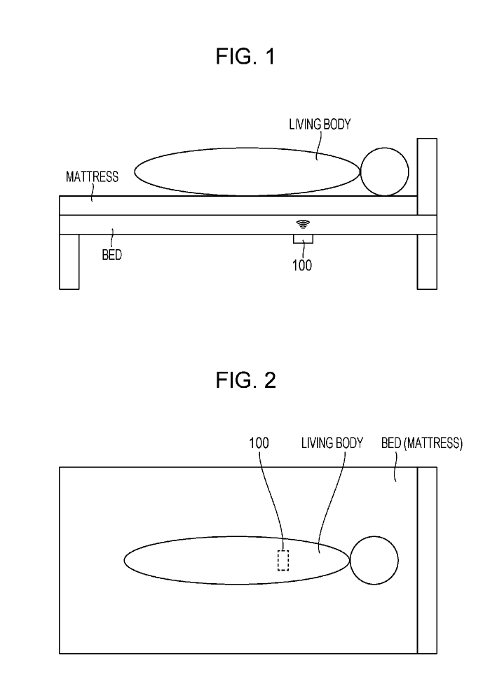

FIGS. 1 and 2 are views each schematically illustrating an. arrangement of the vital sign signal processing device 100 according to the first embodiment. In FIG. 1, a subject (living body) is present on a surface of a bed where a person lie to sleep, and the vital sign signal processing device 100 is disposed on a back surface of the bed FIG. 2 is an upper view of the bed of FIG. 1. The vital sign signal processing device 100 is attached at a location directly below the subject on the bed (see FIG. 2). Note that the vital sign signal processing device 100 may be detachably attachable to the bed.

The vital sign signal processing device 100 includes a battery pack (not shown) which is replaceable or chargeable to supply power to the device. Thus, connection to an external power supply is no longer necessary, which increases the degree of freedom of the installation location. Moreover, the vital sign signal processing device 100 is configured such that the battery pack is replaceable, which enables the vital sign signal processing device 100 to be reusable only by replacing the battery pack even when the power runs out.

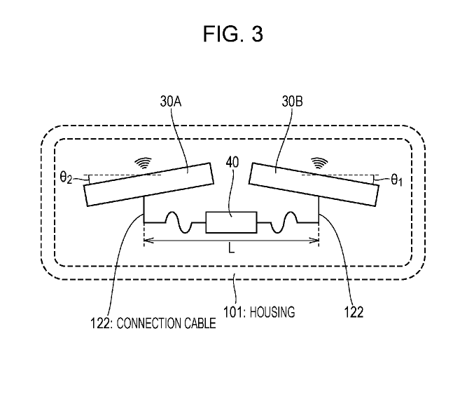

FIG. 3 is a view schematically illustrating a configuration in a housing 101 of the vital sign signal processing device 100 of FIG. 1. In FIG. 3, the vital sign signal processing device 100 includes a housing 101 accommodating a plurality of vital sign signal detection units 30, that is, vital sign signal detection units 30A and 30B, a signal processing unit 40, and cables 122 for wired connection of the vital sign signal detection units 30A and 30B to the signal processing unit 40. Each cable 122 is a retractable communication cable having one end connected to the signal processing unit 40 and the other end connected to the vital sign signal detection unit 30A or 30B. Thus, routing the cable 122 enables the distance L between the signal processing unit 40 and each of the vital sign signal detection units 30A and 30B to be freely changed. Moreover, it is possible to freely chance the relative positional relationship between the signal processing unit 40 and each of the vital sign signal detection units 30A and 30B and the relative positional relationship between the vital sign signal detection units 30A and 30B. A user can freely change attachment angles θ1 and θ2 (positions) of the vital sign signal detection units 30A and 30B in the housing 101. This also enables the above-described relative positional relationship to be adjusted.

The above-described adjustment of the distance L and the angles θ1 and θ2 enables the vital sign signal detection units 30A and 30B to be attached to various beds (e.g., single, double, and reclining beds). This enables antenna directions which will be described later of the vital sign signal detection units 30A and 30B to be adjusted to conform to the shape of a bed.

Note that in FIG. 3, two vital sign signal detection units 30 are accommodated in the housing 101, but three or more vital sign signal detection units may be accommodated.

### Second Embodiment

A vital sign signal processing device 100 according to a second embodiment includes a housing 101 accommodating a signal processing unit 40, and vital sign signal processing device 100 includes a plurality of vital sign signal detection units 30A to 30E disposed outside the housing 101.

Disposing the plurality of vital sign signal detection units 30A to 305 outside the housing 101 increases the degree of freedom of attachment as compared to the first embodiment.

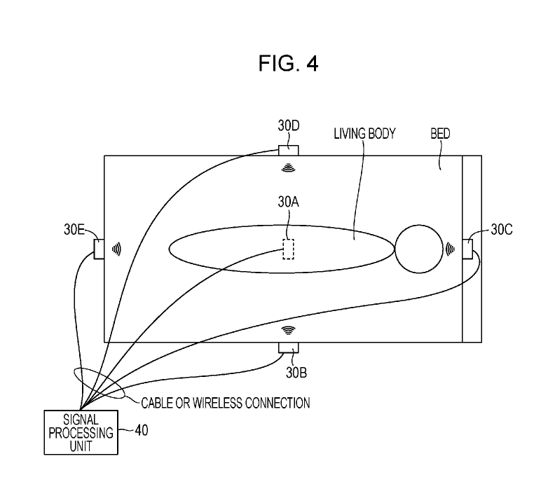

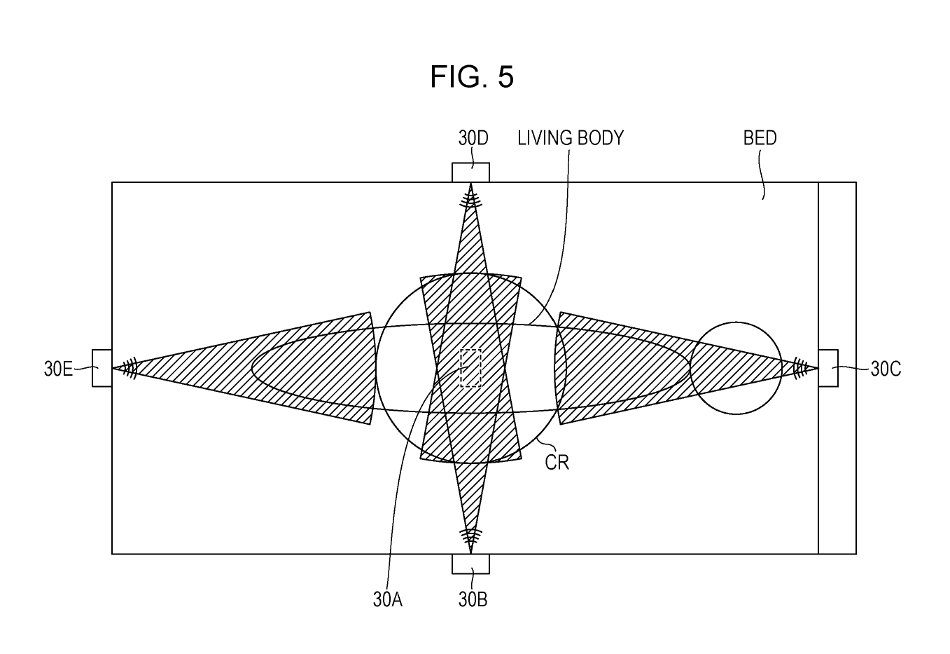

FIG. 4 is a view schematically illustrating attachment of a vital sign signal processing device including a plurality of vital sign signal detection units according to the second embodiment. FIG. 5 is a view schematically illustrating irradiation ranges (see shaded areas in the figure) of radio waves of vital sign signal detection units 30A to 305 of FIG. 4.

In FIG. 4, the vital sign signal detection units 30A to 30E are distributed and disposed at several locations depending on, for example, the size of a bed, that is, in addition to a lower part of the bed directly below a living body (subject), the vital sign signal detection units are disposed above the head and at four corners of the bed.

As described above, it is possible to dispose the vital sign signal detection units 30 in accordance with a condition of a room such as the size of the bed and to increase the range irradiated with a radio wave (see FIG. 5), that is, increase a detection range of the vital sign signal.

In the second embodiment, the plurality of vital sign signal detection units 30A to 30E including antennae communicate with the signal processing unit 40 wirelessly or via lines such as cables. The vital sign signal detection units 30A to 30E and the signal processing unit 40 include wireless communication units (not shown). For the wireless communication, for example, communication using bluetooth (registered trademark), Wi-Fi (registered trademark), Z-WAVE (registered trademark), or the like may be used, but a communication scheme (or protocol) to be applied is not limited to these examples. The wireless connection requires no wiring as compared to wired, cable connection, which provides the degree of freedom of installation of the vital sign signal detection units 30.

In contrast, the wired connection using a cable enables the influence of noise over a communication signal to be eliminated, the communication signal including a detection signal between each vital sign signal detection units 30 and the signal processing unit 40. Moreover, in the wired connection, the vital sign signal detection units 30 do not have to have a wireless transmission function, and thus, cost can be reduced as compared to the wireless connection.

### Third Embodiment

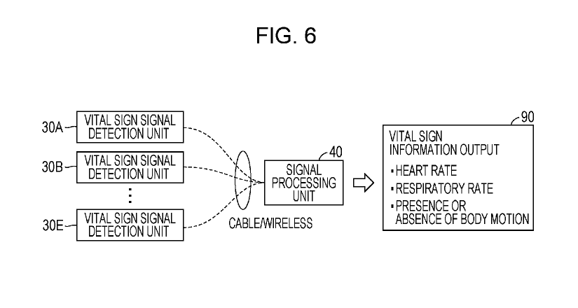

A third embodiment describes a configuration of vital sign signal detection units 30 and a configuration of a signal processing unit 40. FIG. 6 is a view schematically illustrating connection of a vital sign signal processing device 100 and a display unit 90. The display unit 90 includes a liquid crystal display and the like. In this embodiment, the display unit 90 is shown as a device separated from the vital sign signal processing device 100, but the display unit 90 may be provided integrally with the vital sign signal processing device 100.

The display unit 90 displays vital sign information output from the signal processing unit 40. Examples of the vital sign information include the heart rate, the respiratory rate, and the presence or absence of body motion of a living body (subject). Note that a destination to which the vital sign information is output is not limited to the display unit 90, but the destination may include a printing device, an audio output device such as a loudspeaker, a memory device, a server device (including a cloud server), and a communication terminal such as a mobile terminal.

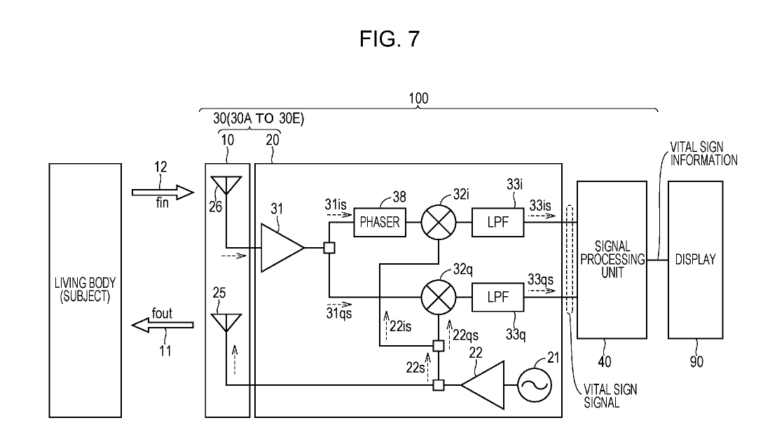

FIG. 7 is a view illustrating an internal configuration of the vital sign signal detection units 30 according to the third embodiment in connection with peripheral elements. A plurality of vital sign signal detection units 30A to 30E are connected to the signal processing unit 40, but to simplify the description, FIG. 7 shows only one vital sign signal detection unit 30 connected to the signal processing unit 40.

(Configuration of Vital Sign Signal Detection Unit 30)

In FIG. 7, the vital sign signal detection unit 30 includes an antenna transmission/reception unit 10 and a detection unit 20. The antenna transmission/reception unit 10 includes a transmit antenna 25 (transmission unit) configured to irradiate the living body with a radio wave (transmit signal 11) and a receive antenna 26 (reception unit) configured to receive a radio wave (reflection signal 12) reflected off the living body. In order to detect the vital sign signal from a reception signal corresponding to the reflection signal 12 received by the receive antenna 26, the detection unit 20 includes an oscillator 21, an amplifier 22, a low-noise amplifier 31, a phaser 38, an I mixer 32i, a Q mixer 32q, and Low Pass Filters (LPFs) 33i, and 33q.

The vital sign signal detection unit 30 of FIG. 7 has a transmission side on which an output signal of the oscillator 21 is amplified by the amplifier 22 and is then transmitted as the transmit signal 11 from the transmit antenna 25. An analog signal 22s which is an output of the amplifier 22 is divided by a distributor into a local oscillation signal 22is on an I side and a local oscillation signal 22qs on a Q side. Note that the oscillator 21 outputs a signal, for example, having a frequency in a 24 GHz band corresponding to a laser radio wave.

The vital sign signal detection unit 30 has a reception side on which the reflection signal 12 as a reflected component of the transmit signal 11 reflected off the living body is amplified via reception antenna 26 by the low-noise amplifier 31 (amplifier which suppresses noise) and is then divided by a distributor into an I analog signal 31is and a Q analog signal 31qs. The phaser 38 receives the I analog signal 31is and outputs to the mixer 32i an analog signal which is a signal obtained by shifting the phase of the I analog signal 31is by 90 degrees. Moreover, the mixer 32q receives the signal 31qs. The phaser 38 causes a phase difference between the I analog signal 31is and the Q analog signal 31qs. These mixers and the LPFs 33i and 33 in the following stage generate a signal 33is of a real part including only a baseband component of the reception signal (corresponding to the reflection signal 12) and a signal 33qs of an imaginary part. The signal 33is and the signal 33qs both correspond to the vital sign signal and are output to the signal processing unit 40.

An output signal (vital sign signal) obtained by inputting the local oscillation signal 22is and the I analog signal 31is on the I side to the mixer 32 includes only a frequency component of a heartbeat, respiration, body motion, or the like. Similarly, an output signal (vital sign signal) obtained by inputting the local oscillation signal 22qs and the Q analog signal 31qs on the Q side to the mixer 32q includes only a frequency component of a heartbeat, respiration, body motion, or the like.

The detection unit 20 detects (extracts) a Doppler shift component included in a reflected wave to obtain a vital sign signal. Thus, even if a radio wave transmitted from another vital sign signal detection unit 30 is received, the influence of the radio wave transmitted from the another vital sign signal detection unit 30 over the vital sign signal to be detected can be reduced.

(Configuration of Signal Processing Unit 40)

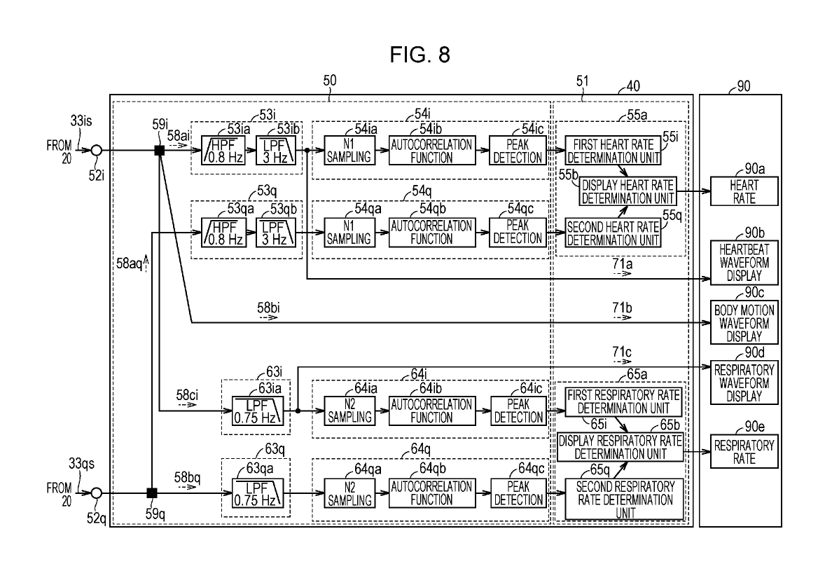

FIG. 8 is a view illustrating a configuration of the signal processing unit 40 of FIG. 7. In FIG. 8, the signal processing unit 40 includes a conversion unit 50 configured to convert an analog vital sign signal received mainly from the vital sign signal detection unit 30 into a signal of digital quantity (hereinafter referred to as data), and an information acquisition unit 51 configured to compute the data after the conversion to acquire vital sign information and outputs the vital sign information to the display unit 90.

The signal processing unit 40 receives from the vital sign signal detection unit 30 the signal 33is (vital sign signal) via an input node 52i and the signal 33qs (vital sign signal) via an input node 52q.

The signal processing unit 40 includes a distributor 59i and a distributor 59g. The distributor 59i is configured to receive and divide the reception signal 33is to output first to third I digital signals 58ai, 58bi, and 58ci. The distributor 59q is configured to receive and divide the reception signal 33qs to output first and second digital signals 58aq and 58bq. The signal processing unit 40 further includes first and second heartbeat signal extraction units 53i and 53q, first and second respiratory signal extraction units 63i and 63q, first and second heartbeat autocorrelation function processing units 54i and 54q, and first and second respiration autocorrelation function processing units 64i and 64q.

The first heartbeat signal extraction unit 53i includes a High Pass Filter (HPF) 53ia and a LPF 53ib which have filter constants for receiving the I digital signal 58ai and extracting a signal (heartbeat waveform signal 71a) of a heartbeat component superimposed on the I digital signal 58ai input to the HPF 53ia. Similarly, the second heartbeat signal extraction unit 53q includes a HPF 53qa and a LPF 53g, which have filter constants for receiving the Q digital signal 58aq and extracting a signal of a heartbeat component superimposed on the Q digital signal 58aq input to the HPF 53ga.

The first respiratory signal extraction unit 63i includes a LPF 63ia having a filter constant for receiving the I digital signal 58ci and extracting a signal (respiration waveform signal 71c) of a respiratory component superimposed on the I digital signal 58ci input to the LPF 63ia. Similarly, the second respiratory signal extraction unit 63q includes a LPF 63qb having a filter constant for receiving the Q digital signal 58bq and extracting a signal of a respiratory component superimposed on the Q digital signal 58bq input to the LPF 63qb.

In order to convert the heartbeat waveform signal output from the first heartbeat signal extraction unit 53i into digital data, the first heartbeat autocorrelation function processing unit 54 includes a sampling processing unit 54ia having a prescribed sampling frequency N1, a first heartbeat autocorrelation function computation unit 54ib, and a peak detection unit 54ic configured to detect the peak value of sampling values. In order to convert the heartbeat waveform signal output from the second heartbeat signal extraction unit 53q into digital data, the second heartbeat autocorrelation function processing unit 54q includes a sampling processing unit 54qa having a prescribed sampling frequency N1, a second heartbeat autocorrelation function computation unit 54qb, and a peak detection unit 54qc configured to detect the peak value of sampling values.

In order to convert the respiratory waveform signal output from the first respiratory signal extraction unit 63i into digital data, the first respiratory autocorrelation function processing unit 64i includes a sampling processing unit 54ia having a prescribed sampling frequency N2, a first respiratory autocorrelation function computation unit 64ib, and a peak detection unit 64ic configured to detect the peak value of sampling values. In order to convert the respiratory waveform signal output from the second respiratory signal extraction unit 63q into digital data, the second respiratory autocorrelation function processing unit 64q includes a sampling processing unit 64qa having a prescribed sampling frequency N2, a second respiratory autocorrelation function computation unit 64qb, and a peak detection unit 64qc configured to detect the peak value of sampling values.

The above-described conversion unit 50 is provided to each of the vital sign signal detection units 30. Thus, when the vital sigh signal detection units 30A to 30E are disposed, conversion units 50 having similar configurations are provided to the respective vital sign signal detection units 30.

The information acquisition unit 51 includes a heart rate determination unit 55a and a respiratory rate determination unit 65a. The heart rate determination unit 55a includes first and second heart rate determination units 55i and 55q and a display heart rate determination unit 55b. Moreover, the respiratory rate determination unit 65a includes first and second respiratory rate determination units 65i and 65q and a display respiratory rate determination unit 65b.

The first heart rate determination unit 55i calculates a central value of a plurality of (M) peak values, for example, sequentially detected by the peak detection unit 54ic. Similarly, the second heart rate determination unit 55q calculates a central value of a plurality of (M) peak values sequentially detected by the peak detection unit 54qc. The first respiratory rate determination unit 65i calculates a central value of a plurality of (N) peak values sequentially detected by the peak detection unit 64ic. Similarly, the second respiratory rate determination unit 65q calculates a central value of a plurality of (M) peak values sequentially detected by the peak detection unit 64qc. Examples of the above-described central values include M median values, maximum values, minimum values, and mode values.

The display heart rate determination unit 55b performs a prescribed computation of the central values from the first and second heart rate determination units 55i and 55q and outputs the resultant value of the computation as display data of a heart rate 90a to the display unit 90. Similarly, the display respiratory rate determination unit 65b performs a prescribed computation of the central values from the first and second respiratory rate determination units 65i and 65q and outputs a resultant value of the computation as display data of a respiratory rate 90e to the display unit 90. Moreover, the display unit 90 receives the heartbeat waveform signal 71a, which is an output signal from the first heartbeat signal extraction unit 53i, a body motion waveform signal 71b which is one of the signals distributed by the distributor 59i, and the respiration waveform signal 71c, which is an output signal from the first respiratory signal extraction unit 63.

The prescribed computation performed by the display heart rate determination unit 55b includes a calculation process of an average value as the central value of heart rates determined by the first and second heart rate determination unit 55i and 55q, but the type of the central value computation is not limited to the average value calculation. For example, when the difference between two heart rates exceeds a threshold value, one of the heart rates which is closer to a predetermined normal value may be determined as the heart rate 90a. Alternatively, instead of the heart rate 90a, or in addition to the heart rate 90a, data of an error display may be output. This computation is performed also by the display respiratory rate determination unit 65b for the respiratory rate 90e in a similar manner.

In the vital sign signal detection unit 30 and the signal processing unit 40, the reflection signal 12 (reception signal) is divided into a real part and an imaginary part to be subjected to signal processes, and process results of both the real part and the imaginary part are used to obtain vital sign information. Thus, even when the vital sign signal is extracted from only the real part (only the imaginary part) depending on the usage environment of the vital sign signal processing device 100, the vital sign information can be obtained and displayed.

### Fourth Embodiment.

In a fourth embodiment, a vital sign signal processing device 100 can be used as a device configured to detect not only vital sign information such as a heart rate, respiratory rate, and body motion but also body motion of a subject on a bed.

Specifically, for motion detection, pattern information denoting a predetermined variation pattern of the vital sign information is stored in memory (not shown). When motion is detected, a signal processing unit 40 compares the vital sign information which is acquired with the pattern information in the memory, and based on the comparison, the signal processing unit 40 determines the variation of the body motion. A result of the determination is displayed by a display unit 90.

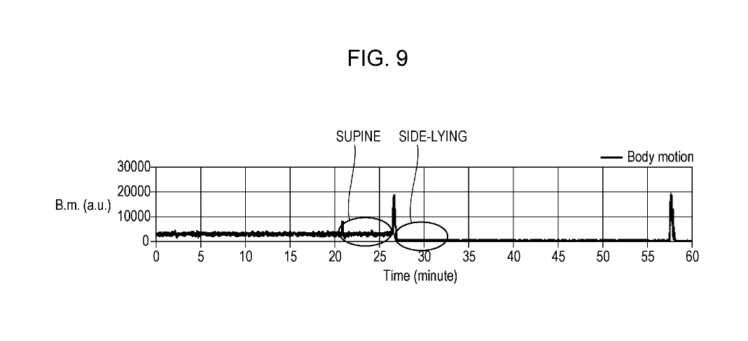

FIG. 9 is a graph illustrating an example of a body motion waveform signal 71b according to the fourth embodiment. In the graph, the abscissa denotes a time lapse, and the ordinate denotes values of amplitude.

The signal processing unit 40 compares a time-sequence vital sign signal which is from a vital sign signal detection unit 30 and which is shown in FIG. 9 with the pattern information predetermined and stored in the memory. The pattern information includes a variation in the magnitude of the amplitude of the vital sign signal detected when the subject rolls over on the bed, for example, when the subject rolls from a supine state to a side-lying state. The signal processing unit 40 determines the occurrence of rolling over when a partial signal which matches to the pattern information is detected in the body motion waveform signal 71b detected in time sequence. Moreover, it is possible to detect a time when the subject rolls over based on a time when the partial signal is detected in time sequence. As illustrated in FIG. 9, it is also possible to identify a time when the subject is in a position (supine position, side-lying position, or the like) on the bed.

As a variation, the pattern information may include pattern information of a body motion signal denoting whether or not the subject is on the bed. In this case, in a manner similar to the above-described manner, it is possible to perform motion detection that the subject leaves the bed for is on the bed) and to detect a time when the subject leaves the bed or a time when the subject is on the bed. Moreover, pieces of the pattern information are measured for each subject and type (rolling over, leaving the bed, being on the bed, or the like) of motion (body motion) and are stored in the memory in advance, which enables determination specific to the subject to be made for each types of motion.

Note that in the embodiment, the signal processing unit 40 uses the body motion waveform signal 71b to detect the above-described body motion, but the vital sign signal used is not limited to the body motion waveform signal 71b. For example, when the attention is focused on that the heartbeat waveform signal 71a or the respiration waveform signal 71c changes depending on the location (movement) of the chest, nose, and/or mouth of the living body, whether or not the subject is on the bed, that is, movement of, for example, leaving the bed or returning to the bed can be determined in accordance with the presence or absence of the heartbeat waveform signal 71a or the respiration waveform signal 71c.

Moreover, when the heartbeat waveform signal 71a, the body motion waveform signal 71b, and the respiration waveform signal 71c are combined with each other to determine the position and/or movement of the subject on the bed, determination accuracy can be increased.

### Fifth Embodiment

The above-described signal processing unit 40 may be realized by a program executed by a hardware circuit or a processor as described in a fifth embodiment. Alternatively, the signal processing unit 40 can be realized by a combination of both the hardware circuit and the program.

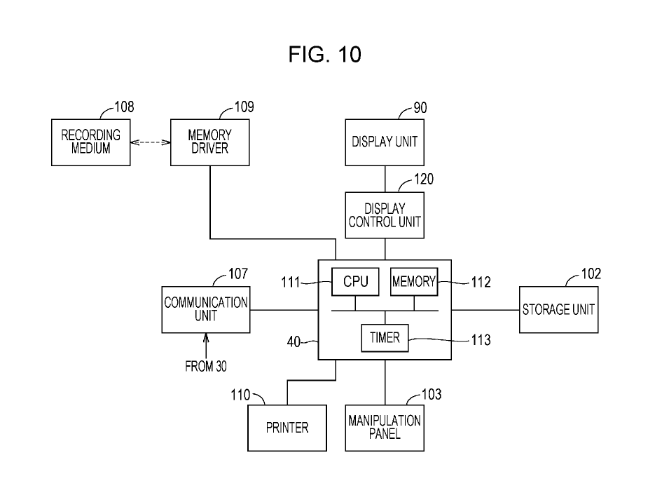

FIG. 10 is a block diagram illustrating a signal processing unit 40 according to the fifth embodiment in connection with peripheral elements. In FIG. 10, the signal processing unit 40 includes a computer including a processor. Specifically, the signal processing unit 40 the signal processing unit 40 includes Central Processing Unit (CPU) 111, memory 112, and a timer 113. The signal processing unit 40 is connected to a storage unit 102 configured to store a program, data, and the like, a manipulation panel 103 configured to receive an input to the signal processing unit 40 by a user, a communication unit 107 configured to communicate with external devices including a vital sign signal detection unit 30, a memory driver 109 to which a recording medium 108 is detachably attached externally and which is configured to read and write data about the recording medium 108 which is attached, a printer 110, and a display control unit 120 configured to control the display unit 90. Note that the display unit 90 and the manipulation and 103 may be integrated with each other to be provided as a tablet device.

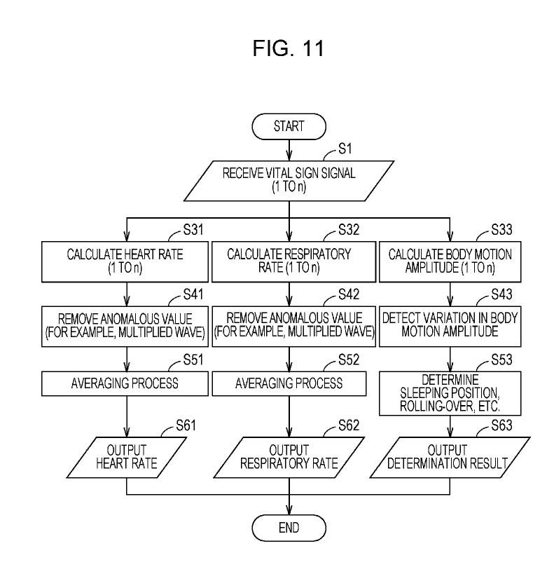

FIG. 11 is a flow chart of processes performed by the signal processing unit 40 according to the fifth embodiment. The flow chart of the processes is stored as a program in the memory 112 or the recording medium 108 of FIG. 10. The CPU 111 reads a program from the storage units and executes the program which is read. In FIG. 11, the signal processing unit 40 communicates with n vital sign signal detection units 30. Moreover, the pattern information described in the fourth embodiment is stored in the memory 112 or the recording medium 108.

In FIG. 11, the vital sign signal processing device 100 is activated when a subject sleeps. First, the CPU 111 receives vital sign signals from the n vital sign signal detection units 30 (step S1). Thus, n vital sign signals are obtained.

Based on the n vital sign signals, the CPU 111 concurrently performs an acquisition process of a heart rate 90a (step S31 to step S61), an acquisition process of a respiratory rate 90e (step S32 to step S62), and a determination process of the body motion described in the fourth embodiment (step S33 to step S63).

Specifically, in the acquisition process of the heart rate 90a, the heart rate is calculated in a similar manner to the first and second heart rate determination units 55i and 55q (step S31). The CPU 111 removes an anomalous value included in the heart rate which is calculated (step S41) and then, from the data of the heart rate, the CPU 111 determines a heart rate to be displayed by an averaging process in a similar manner to the display heart rate determination unit 55b (step S51) and outputs the heart rate 90a which is determined to the display unit 90 via the display control unit 120.

Moreover, in the acquisition process of the respiratory rate 90e, the respiratory rate is calculation in a similar manner to the first and second respiratory rate determination units 65i and 65q (step S32). The CPU 111 removes an anomalous value included in the respiratory rate which is calculated (step S42), and then, from the data of the respiratory rate, the CPU 111 determines a respiratory rate to be displayed by an averaging process in a similar manner to the display respiratory rate determination unit 65b (step S61) and outputs the respiratory rate 90e which is determined to the display unit 90 via the display control unit 120.

Moreover, for the determination process of the body motion, the CPU 111 includes a body motion determination process unit. The body motion determination process unit performs the process described in the fourth embodiment. In the present embodiment, a determination process based on a body motion waveform signal 71b from n vital Sign signal detection units 30 is described. First, a waveform amplitude of each of the n body motion waveform signals 71b which are contiguous in waveform in time sequence is detected (step S33), and a time-sequence variation of the amplitude detected in time sequence is detected (step S43). Then, the body motion determination process unit compares information about the time-sequence amplitude variation (see FIG. 9) detected for each body motion waveform signal 71b with pattern information denoting a pattern of the amplitude variation of rolling over stored in, for example, the memory 112, and based on a result of the comparison, the body motion determination process unit determines whether or not the time-sequence amplitude variation includes the pattern (see FIG. 9) of the amplitude variation of the rolling over (step S53). For example, when the pattern information denoting the rolling over is detected in a majority of n body motion amplitudes, the body motion determination process unit outputs a determination result denoting detection of the rolling over to the display unit 90 via the display control unit 120.

Here, removal of the anomalous value in steps S41 and S42 will be described. Removing the anomalous value enables a highly accurate signal to be selected as a vital sign signal for calculation (acquisition) of the heart rate and/or respiratory rate.

In a method for removing the anomalous value, for example, a threshold value (e.g., normal value) corresponding to each of the heart rate and the respiratory rate is registered, and the CPU 111 compares each of the n heart rates (or n respiratory rates) acquired via the vital sign signal detection units 30 with a corresponding one of the threshold values. Then, based on a result of the comparison, the CPU 111 determines a value of the n heart rates (or n respiratory rates) which is to be a target (a value which is to be excluded from a target) subjected to the averaging process. For example, the CPU 111 excludes and discard a heart rate (or a respiratory rate) exceeding the threshold value from the target of the averaging process. Note that exceeding the threshold value means being higher than a normal value or being lower than the normal value.

Alternatively, the CPU 111 excludes a value which is included in but significantly different from values of the n heart rates (or n respiratory rates) from the target of the averaging process. Note that it is desirable that the threshold value be registered for each subject.

The anomalous value is attributed to noise which may be included in a vital sign signal in the vital sign signal detection unit 30. Specifically, when vital sign signals are extracted from the I mixer 32i and the Q mixer 32q, a harmonic component is generated in addition to a vital sign signal component which is necessary. Depending on a harmonic frequency, noise cannot be removed by filters (LPFs 33i and 33q) in the following stage, and the frequency component is mixed as noise in the vital sign signal. For example, when the body motion is small, the heartbeat waveform signal 71a which is detected includes a lot of multiplied waves such. as double-multiplied waves or triple-multiplied waves, and the multiplied waves may result in the noise.

In step S41, vital sign information obtained from a vital sign signal which includes no multiplied wave and which is included in a plurality of vital sign signals extracted from the reception signals of the plurality of receive antennas 26 is used to perform the averaging process, which enables the calculation accuracy of the heart rate and/or the respiratory rate to be increased.

It should be understood that the embodiments disclosed herein have been described for the purpose of illustration only and in a non-restrictive manner in any respect. The scope of the present invention is defined by the terms of the claims, rather than the description above, and is intended to include any modifications within the meaning and scope equivalent to the terms of the claims.

### REFERENCE SIGNS LIST

30, 30A to 30E VITAL SIGN SIGNAL DETECTION UNIT

40 SIGNAL PROCESSING UNIT

90 DISPLAY UNIT

100 VITAL SIGN SIGNAL PROCESSING DEVICE

1. A vital sign signal processing device comprising:
a plurality of vital sign signal detection units configured to detect vital sign signals of a subject; and
a signal processing unit configured to process the vital sign signals detected by the plurality of vital sign signal detection units and to acquire vital sign information denoting a vital phenomenon which is movement of a living body, wherein
each of the plurality of vital sign signal detection units includes
a transmission unit configured to transmit a radio wave with which the living body is irradiated and
a reception unit configured to receive a signal corresponding to a radio wave reflected off the living body,
each of the plurality of vital sign signal detection units is configured to detect the vital sign signal from the signal received by the reception unit,
each vital sign signal includes a heartbeat signal denoting a heartbeat or a respiratory signal denoting respiration.
the vital sign information includes heart rates or respiratory rates, and
the signal processing unit is configured to discard a heart rate or a respiratory rate of the heart rates or the respiratory rates acquired from the vital sign signals which exceeds a threshold value.

2. (canceled)

3. The vital sign signal processing device according to claim 1, wherein
the signal processing unit is configured to calculate a central value of the heart rates or the respiratory rates acquired from the vital sign signals.

4. The vital sign signal processing device according to claim 1, wherein
the signal processing unit is configured to perform comparison of pattern information denoting a pattern of a predetermined change of vital sign information of the subject with the vital sign information acquired from the vital sign signals which are detected so as to determine body motion which is body movement of the subject in accordance with the comparison.

5. The vital sign signal processing device according to claim 4, wherein
the vital sign signal detection unit is configured to detect the vital sign signals in time sequence, and
the signal processing unit is configured to determine a time when the body motion occurs in time sequence in accordance with the comparison.

6. A vital sign signal processing device comprising:
a plurality of vital sign signal detection units disposed at a lower portion of a bed of a subject and configured to detect vital sign signals of the subject; and
a signal processing unit configured to process the vital sign signals detected by the plurality of vital sign signal detection units and to acquire vital sign information denoting a vital phenomenon which is movement of a living body, wherein
each of the plurality of vital sign signal detection units includes
a transmission unit configured to transmit a radio wave with which the living body is irradiated and
a reception unit configured to receive a signal corresponding to a radio wave reflected off the living body,
each of the plurality of vital sign signal detection units is configured to detect the vital sign signal from the signal received by the reception unit, and
the vital sign signal processing device includes a distance adjustment means by which a distance between the plurality of vital sign signal detection units is adjustable.

7. A vital sign signal processing device comprising:
a plurality of vital sign signal detection units disposed at a lower portion of a bed of a subject and configured to detect, vital sign signals of the subject; and
a signal processing unit configured to process the vital sign signals detected by the plurality of vital sign signal detection units and to acquire vital sign information denoting a vital phenomenon which is movement of a living body, wherein
each of the plurality of vital sign signal detection units includes
a transmission unit configured to transmit a radio wave with which the living body is irradiated and
a reception unit configured to receive a signal corresponding to a radio wave reflected off the living body,
each of the plurality of vital sign signal detection units is configured to detect the vital sign signal from the signal received by the reception unit, and
the vital sign signal processing device includes an angle adjustment means by which an attachment angle of the plurality of vital sign signal detection units is adjustable.

8. A vital sign signal processing device comprising:
a plurality of vital sign signal detection units disposed at a lower portion of a bed of a subject and configured to detect vital sign signals of the subject; and
a signal processing unit configured to process the vital sign signals detected by the plurality of vital sign signal detection units and to acquire vital sign information denoting a vital phenomenon which is movement of a living body, wherein
each of the plurality of vital sign signal detection units includes
a transmission unit, configured to transmit a radio wave with which the living body is irradiated and
a reception unit configured to receive a signal corresponding to a radio wave reflected off the living body,
each of the plurality of vital sign signal detection units is configured to detect the vital sign signal from the signal received by the reception unit, and
each of the plurality of vital sign signal detection units and the signal processing unit are connected to each other via a retractable ca
